(19) United States
(12) Patent Application Publication
(10) Pub. No.: US 2022/0175594 A1
(43) Pub. Date: Jun. 9, 2022

(54) DEVICES AND METHODS FOR ELEVATED CARDIOPULMONARY RESUSCITATION

(71) Applicant: MICHAEL FONTE, HOLLYWOOD, FL (US)

(72) Inventor: MICHAEL FONTE, HOLLYWOOD, FL (US)

(21) Appl. No.: 17/678,546

(22) Filed: Feb. 23, 2022

Related U.S. Application Data
(63) Continuation-in-part of application No. 17/217,129, filed on Mar. 30, 2021.
(60) Provisional application No. 63/153,036, filed on Feb. 24, 2021.
(60) Provisional application No. 63/002,563, filed on Mar. 31, 2020.
(60) Provisional application No. 63/153,036, filed on Feb. 24, 2021.

(51) Int. Cl. A61G 1/044; A61M 16/00; A61H 31/00

(52) U.S. Cl. CPC A61G 1/044; A61M 16/0048; A61G 2200/327; A61G 2210/00; A61H 31/008

(57) ABSTRACT

Devices and methods for performing elevated cardiopulmonary resuscitation are provided. A device includes a mechanism for changing the angle of elevation of a backrest of a patient support to elevate the head and thorax of a patient during elevated or head-up CPR. The device additionally includes, stored in a non-transitory memory device thereof, a program for performing elevated CPR that, when executed by a processor of the device acts to elevate the head and thorax of a patient according to a predetermined plan.

### CROSS-REFERENCE TO RELATED APPLICATIONS

The present application claims the benefit of Provisional Patent Application No. 63/153,036, filed on Feb. 24, 2021, entitled Devices and Methods for Elevated Cardiopulmonary Resuscitation; and from U.S. patent application Ser. No. 17/217,129, filed on Mar. 30, 2021, entitled Elevated CPR Assist Device and Method, which application claims the benefit of Provisional Patent Application No. 63/002,563, filed on Mar. 31, 2020 entitled Elevated CPR Assist Device, and Provisional Patent Application No. 63/153,036, filed on Feb. 24, 2021; the contents of those applications being incorporated herein, by reference, in their entireties.

### BACKGROUND OF THE INVENTION

### Field of the Invention

The invention relates generally to devices and a methods that aid in performing cardiopulmonary resuscitation (CPR) and, more particularly, to methods and devices useful for performing elevated or “head-up” CPR which assist the medical personnel or person conducting the elevated CPR method in easily, rapidly and precisely elevating the subject to the predefined positions or elevations required by the method.

### DESCRIPTION OF THE RELATED ART

Cardiopulmonary Resuscitation (CPR) is a medical procedure which involves performing repeated compression of a patient's chest in an attempt to restore the blood circulation and breathing of the patient during cardiac arrest. For adults, CPR may involve chest compressions of between 5-6 cm (2-2.4 inch) deep and at a rate of at least 100-120 compressions per minute. Artificial ventilation may simultaneously be provided by either exhalation of air into the subject's mouth or nose, in the case of mouth-to-mouth resuscitation, or by use of mechanical ventilation, in which a device is used to push air into the subject's lungs. The main objective of CPR is to restore partial flow of oxygenated blood to the brain and heart of the patient in order to delay tissue death and extend the brief window of opportunity for a successful resuscitation without permanent brain damage.

The elevated CPR method is a technologically advanced technique for performing device-assisted head-up CPR. The method incorporates raising the head, heart and thorax in a multi-level elevation, in a timed sequence and in conjunction with best practice resuscitation protocols. Implementation of the CPR procedure on a patient with the patient's head, heart and thorax at these elevated positions may help relieve pressure on the brain of the patient. The timing and elevation sequence for the elevated CPR method are scientifically based on preclinical studies which have shown the benefits of a sequence that includes a two minute period of high quality CPR with ACD/ITD or mCPR/ITD with the patient in low supine position followed by a slow progressive rise of 6 cm/min over two minutes to a head height of 22 cm and a thorax of 8 cm.

Preclinical studies have shown that the addition of the elevated CPR method to the existing use of an ITD and ACD-CPR or mechanical CPR significantly increases cerebral perfusion pressure to greater than 80% of normal when two minutes of priming in the lowered position preceded slowly raising the head and thorax of the patient to elevated positions over a two-minute period. The elevated CPR method has been shown to immediately and significantly decrease intracranial pressure while maintaining aortic pressure, reducing the chance of a brain concussion with every compression. Additionally, the elevated CPR method has been shown to significantly increase coronary perfusion pressure to greater than 70% of normal when two minutes of priming in the lowered position preceded slowly raising of the head and thorax to the elevated positions over a two-minute period.

There remains a need in the art, however, for solutions which facilitate conducting the elevated CPR method, and more specifically, for solutions which assist the medical personnel or person conducting the elevated CPR method in easily, rapidly and precisely elevating the subject to the predefined positions or elevations required by the method.

### SUMMARY OF THE INVENTION

It is accordingly an object of the invention to provide devices and methods for elevated CPR that addresses the needs set forth above. For example, the invention is directed an elevated CPR assist device suitable to elevate the head and thorax of a patient while elevated CPR is carried out on the patient.

Particular methods for using a head-up CPR device in accordance with the present application are additionally provided.

Other features which are considered as characteristic for the invention are set forth in the drawings, description, and appended claims.

Although the invention is illustrated and described herein as embodied in devices and methods for elevated cardiopulmonary resuscitation, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings, in which like reference numerals represent like items.

### BRIEF DESCRIPTION OF THE DRAWING

FIG. 1 is a partial, perspective view of a stretcher having a head and thorax elevating device in accordance with one particular embodiment of the invention retrofit thereto;

FIG. 2 is a partial, top plan view of the stretcher of FIG. 1 having the head and thorax elevating device in accordance with one particular embodiment of the invention retrofit thereto;

FIG. 3A is a side plan view of a stretcher including a head elevating device in accordance with one particular embodiment of the invention, wherein the head elevating device is in its fully retracted first position;

FIG. 3B is a side plan view of a stretcher including a head elevating device in accordance with one particular embodiment of the invention, wherein the head elevating device is in an extended second position;

FIG. 4 is a cross-sectional view from the side of a head elevating device in accordance with one particular embodiment of the present invention;

FIG. 5 is a simplified block diagram of a head elevating device in accordance with one particular embodiment of the invention;

FIG. 6 is a partial, perspective view of a head elevating system including a stretcher having a built-in head elevating device in accordance with one particular embodiment of the invention;

FIG. 7 is a partial, top plan view of the stretcher of FIG. 5 having a built-in head elevating device in accordance with one particular embodiment of the invention;

FIG. 8 is a partial, side plan view of a stretcher including a built-in head elevating mechanism in accordance with one particular embodiment of the invention, wherein the head elevating mechanism is shown having elevated the stretcher backrest;

FIG. 9 is a simplified block diagram of a head elevating mechanism in accordance with one particular embodiment of the invention;

FIG. 10 is a perspective view of a portable head elevating device in accordance with one particular embodiment of the invention;

FIG. 11 is a perspective view of the head elevating device of FIG. 10, folded up for carrying;

FIG. 12 is a perspective view of the portable head elevating device of FIG. 10, with a head elevation mechanism in accordance with one particular embodiment deployed;

FIG. 13 is a perspective view of another embodiment of a portable head elevating device;

FIG. 14 is a perspective view of a further embodiment of a portable head elevating device;

FIG. 15 is a simplified block diagram of a head elevating device in accordance with one particular embodiment of the invention;

FIG. 16, is a partial, side plan view of a stretcher having a head elevating device in accordance with one particular embodiment of the invention retrofit thereto;

FIG. 17 is a front view of a portion of the head elevating device of FIG. 16, showing additional buttons for controlling the motor;

FIG. 18 is a partial, side sectional view of the head elevating device of FIG. 16, in which the motor and gears can be seen;

FIG. 19 is a partial, front cut-away view of a head elevating device in accordance with another particular embodiment of the present invention; and

FIG. 20 is a simplified flow diagram of a method in accordance with one particular embodiment of the invention.

### DETAILED DESCRIPTION OF THE INVENTION

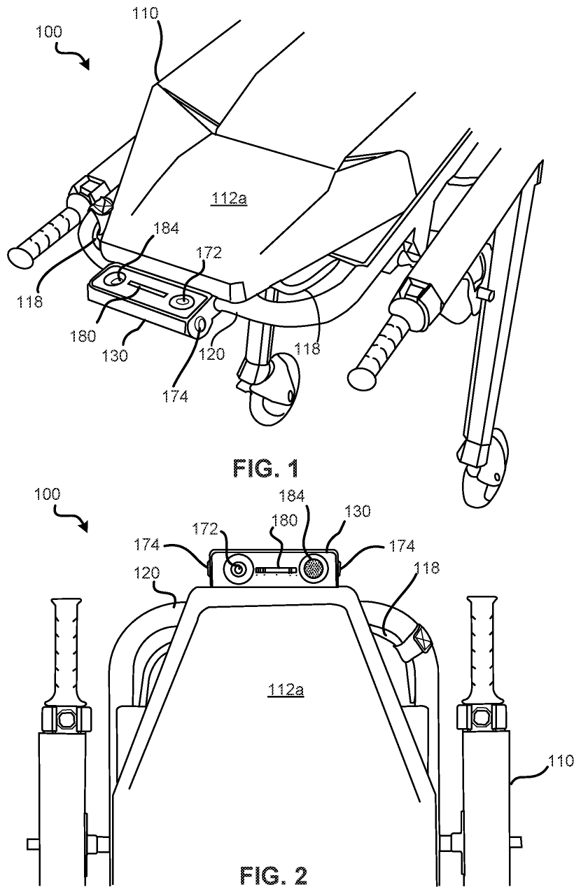

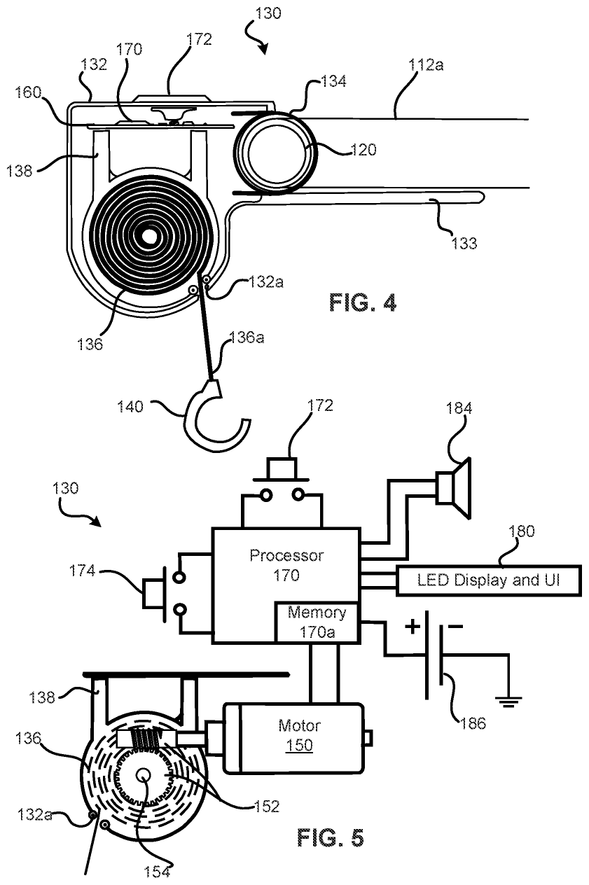

Referring now to FIGS. 1-5, there is shown a system 100 including an emergency stretcher or ambulance cot 110 and a head (and thorax) elevating CPR assist device 130 mounted to an emergency stretcher 110. In particular, the head elevating CPR assist device 130 is configured to be added to, or retrofit to, an existing stretcher 110. For example, the head elevating CPR assist device 130 of the present embodiment can be used with any type of emergency stretcher, including, but not limited to, the stretchers described in U.S. Pat. Nos. 5,537,700 and 6,735,794, and United States Patent Application Publication Nos. 2009/178200 and 2019/0192365, those four references being incorporated herein by reference in their entireties. Those references show only exemplary emergency stretchers with which the invention described in connection with FIGS. 1-5 can be used. It should be understood that the invention can be used with other types of stretchers and ambulance cots that include a backrest movable relative to a base without departing from the scope or spirit of the present invention.

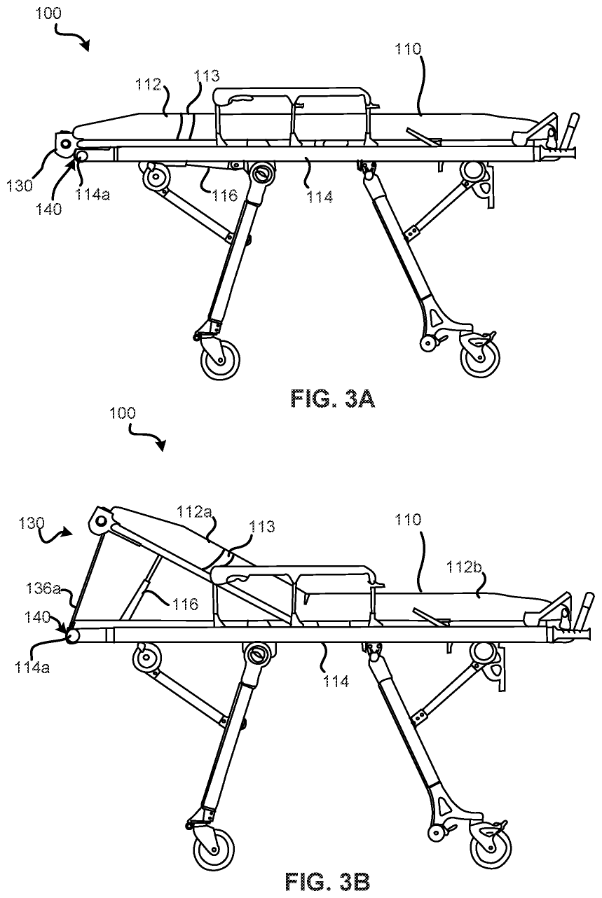

Referring back to FIGS. 1-5, the stretcher 110 includes a patient support 112 that is mounted relative to a base frame 114, and which supports an upper body support member or backrest 112a that tilts relative to the base frame 114. For example, the angle of elevation of the backrest 112a of patient support 112 can be changed relative to the lower body support member 112b of the patient support 112 and/or relative to the base frame 114 (see FIG. 3B), thus, tilting the head and back of the patient relative to the patient's lower body. Typically, the base frame 114 is oriented parallel to the ground and the backrest 112a inclines relative to a plane parallel to the ground. The mechanism for changing the angle of elevation of the backrest portion 112a of the patient support 112 will be specific to the stretcher. However, in the present particular embodiment illustrated, a telescoping pneumatic piston or gas cylinder 116 is used to raise (i.e., increase the angle of elevation) of the backrest 112a relative to the base frame 114, when a backrest adjustment handle or lever 118 is actuated. When the adjustment handle 118 is manually actuated, the piston 116 permits the backrest 112a of the patient support 112 to pivot relative to the base frame 114.

The head elevating device 130 of FIGS. 1-5 is designed as a self-contained unit to be added after the fact (i.e., retrofitted) to an existing stretcher. Note however, the invention is not meant to be limited only thereto, as the head elevating device 130 can be made as part of a stretcher 110, without departing from the scope or spirit of the present invention. In the present embodiment, the housing 132 of the head elevating device 130 is attached to an end frame 120 of the backrest 112a using a hose clamp 134. An anti-rotation plate 133 of the housing 132 stabilizes the head elevating device 130 on the backrest 112a.

Inside the housing 132, the head elevating device 130 includes a spring-loaded nylon webbing coil or strap 136 mounted on a reel or spindle 154 in the housing 132, by a frame 138. A leading end or free end 136a of the nylon webbing exits the housing 132 through a slit 132a. The spring-loaded nylon webbing coil 136 can be of a type used in connection with automobile seatbelts, if desired. An attachment device 140 is connected at a free end of the leading edge 136a, outside the housing 132, for clamping the web 136 to the head end lifting bar 114a of the base frame 114. The attachment device can be any mechanism that secures the free end of the nylon web around the head end lifting bar, including, but not limited to, a hook, a clamp, a strap, etc. In the present embodiment illustrated, the attachment device includes a hook 140. Other attachment devices can be used to secure the free end of the web 136 to the base frame 114 (e.g., snaps, hook and loop fasteners, etc.), in addition to, or instead of, the hook 140, as desired. A motor 150, contained inside the housing 132, is provided to wind and unwind the strap or webbing coil 136 in accordance with a program for elevated CPR stored in memory 170a, and executable by the processor 170, as will be described below. Gearing or gear train 152 may be provided to drive a reel or spindle 154 containing the web coil 136. Alternately, a spindle of the motor 150 can be used to directly wind and unwind the spring-loaded nylon webbing coil 136, as desired.

The head elevating device 130 additionally includes a printed circuit board (PCB) 160 mounted within the housing 132 which receives power from a power source 186 that can be a battery provided internal to, or external to, the housing 132. Alternately, an on-board power supply of stretcher 110 can be used to power the head elevating device 130. A processor 170 is mounted on the PCB and programmed to operate the head elevating device 130 in order to control the angle of elevation of the backrest 112a during CPR, tilting the backrest 112a at particular angles relative to the frame 114, for example, 10, 20, 30 degrees, automatically, and hold the backrest 112a at each angle for a period of time defined by the stored program. An on/off button 172 is accessible from outside the housing 132, to turn the device 130 on or off, via its integration with elements on the PCB 160. Operation of the on/off button 172 provides power to the device 130 and initiates execution of a CPR program, stored in a non-transitory memory 170a, by the processor 170 of the head elevating device 130. One or more override or emergency off switches 174 are provided which are accessible from the side(s) of the housing 132, in order to override the program being executed by the processor 170 and to quickly turn off the device 130, if desired.

The processor 170 is additionally configured to control a user interface (UI) for providing clear feedback to the operator. For example, the processor 170 can be configured to provide an audible and/or visual feedback to the operator for each stage of use. For this purpose, the head elevating device 130 can be provided with a visual indicator and user interface (UI) 180 and a speaker 184, the outputs to which are controlled by the processor 170. For example, the visual indicator and UI 180 can include one or more light emitting diodes (LEDs), controlled by the processor 170, to provide a visual confirmation to the operator at certain stages of use. Additionally, the processor 170 provides audible confirmation to the operator via the speaker 184 for each stage of use.

Among other things, the visible and audible indicators are provided to the operator when the device is turned on, as well as for the ongoing status and to assist with other operations. Additionally, indicators (audio or visual) may be provided: 1) to confirm activation of the unit; 2) at each pass of the backrest being raised; 3) when the maximum backrest height has been reached; and 4) after 10 minutes have passed, to switch to automatic CPR. The foregoing is not meant to be limiting, as other indicators may be provided instead of, or in addition to, those listed herein above.

In use, the housing 132 of the head elevating device 130 is attached to the frame 120 of the head end of the backrest 112a of the patient support 112, while the free end 136a of the web coil 136 is attached to the fixed frame of the stretcher 110, via the attachment device 140. In one particular example, an attachment device 140 is used to secure the free end of the web 136a to the head end lifting bar 114a of the base frame 114 of the stretcher 110. A lever strap 182 is used to pull the backrest adjustment handle or lever 118 and secure it to the frame 120 of the backrest 112a, in the pulled position. This releases the gas cylinder 116. The backrest 112a, however, does not rise as it normally would when the backrest adjustment lever 118 is pulled. Rather, the motor 150 maintains a tension on the spring-loaded nylon webbing coil 136, to maintain the free end 136a at its shortest length, and keep the backrest 112a horizontal (see, for example, FIG. 3A). The motor 150 will operate to periodically unwind the webbing coil 136, to allow the gas cylinder 116 to raise the backrest 112a upward and/or downward in defined steps and hold it at a given position for defined periods of time, in accordance with the program for elevated CPR executed by the processor 170. See, for example, FIG. 3B. More particularly, the device includes a program for performing elevated CPR stored in a non-transitory memory device 170a that, when executed by the processor 170, acts to elevate the head and thorax of a patient according to a predetermined plan for head elevated CPR.

In particular, when elevating the patient's torso, the device operates to elevate the patient's head so that it rests slightly higher than the torso. In one particular non-limiting example, the program for head elevation elevates the head to a minimum of 1.0-1.5 inches above the torso (i.e., relative to a horizontal starting plane), at the lowest point, up to 6 inches maximum (i.e., also relative to the horizontal plane), over a 2 minute span of time. The 2 minute time span prevents the patient's head from being raised too quickly, which could otherwise cause the patient's blood pressure and intra-cranial pressure to decrease too rapidly. A program for performing elevated CPR that can be used is disclosed in U.S. Pat. No. 10,350,137 to Lurie, entitled Elevation Timing Systems and Methods for Head Up CPR, which patent is incorporated herein, by reference, in its entirety.

Similarly, the motor 150 can be used to wind the web coil 136, in order to lower the backrest 112a against the force applied by the gas piston 116, while the lever strap 182 holds the backrest adjustment lever 118 in the open (pulled) position. Removal of the lever strap 12 permits the lever 116 to return to its normal position, again locking the backrest 112a into its current position. A mechanism, such as strap 113, can be provided on the stretcher 110 for holding the patient and preventing the patient from sliding down when the maximum elevation is reached.

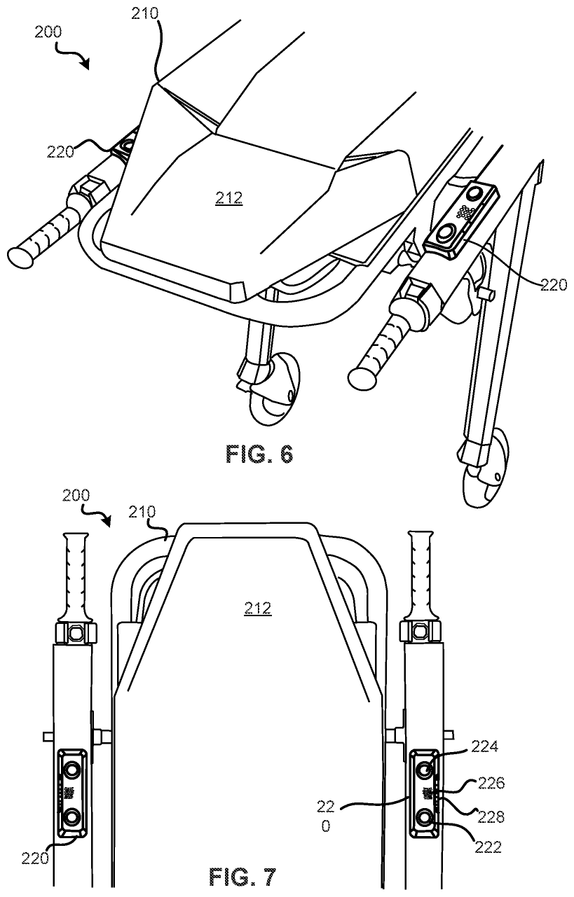

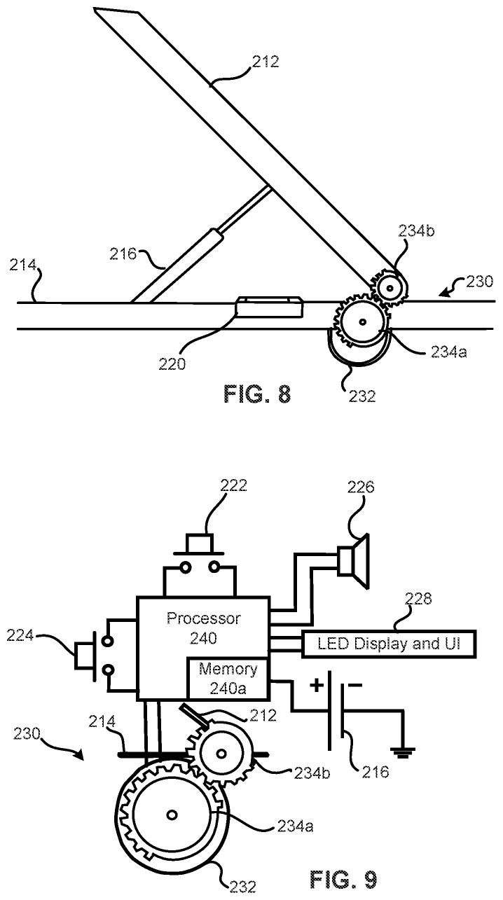

Referring now to FIGS. 6-9, another embodiment of head elevated device 200 for CPR is illustrated. More particularly, a stretcher 210 is provided having head elevation controls 220 and operational components 230 integrated into the stretcher 210. The head elevation controls 220 are powered from a power supply 216 of the stretcher 210. The operational components 230 include a motor 232 and gearing or gear train 234a, 234b for elevating the backrest 212 relative to a frame 214 of the stretcher 210. The head elevation controls 220 permit actuation of the operational components 230, to elevate and lower the backrest 212 in accordance with a program stored in memory 240a and executed by a processor 240 of the head elevation controls 220. As with the previously described embodiment, the head elevation controls 220 include an on switch 222, an off switch/override 224 a speaker 226 and an LED user interface 228. A processor 270 associated with the controls 220 is configured to execute a program for performing elevated CPR, stored in memory 270a, once being initiated by activation of the on switch 222. In particular, the internal processor 270 controls the motor 232 to spin, thus turning a first gear 236 attached to the spindle of the motor 232. First gear 234a engages a second gear 234b fixed to the frame of the backrest 212, to raise or lower the backrest 212 relative to the stretcher frame 214 (assisted by the gas cylinder 216) in accordance with a preset CPR program for performing elevated CPR, stored in a non-transitory memory 270a of the device 200 that, when executed by a processor of the device acts to elevate the head and thorax of a patient according to a predetermined plan. The CPR program can operate as described in connection with the embodiment of FIGS. 1-5, providing audible and visual indicators to an operator, and elevating or lowering (i.e., increasing or decreasing the angle of elevation of) the backrest 212, in order to position the head and thorax of a patient at a desired height for desired periods of time for elevated CPR.

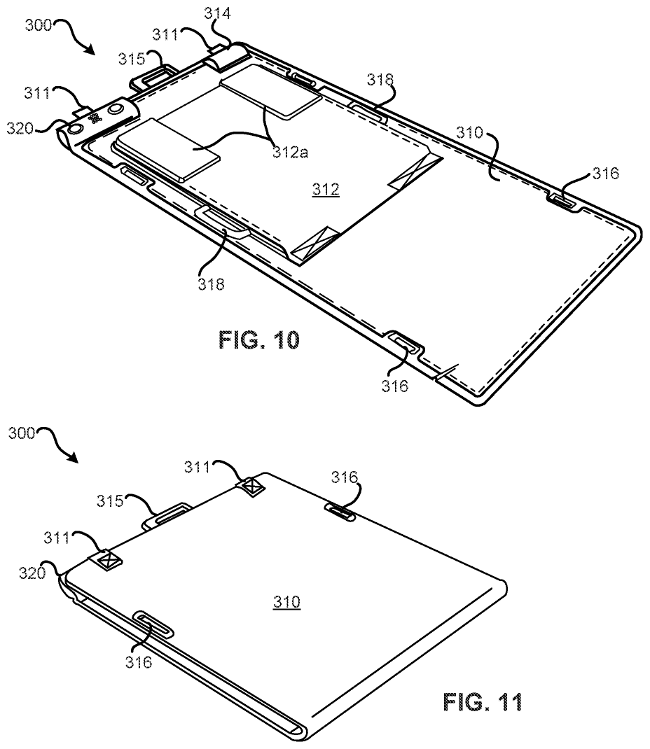

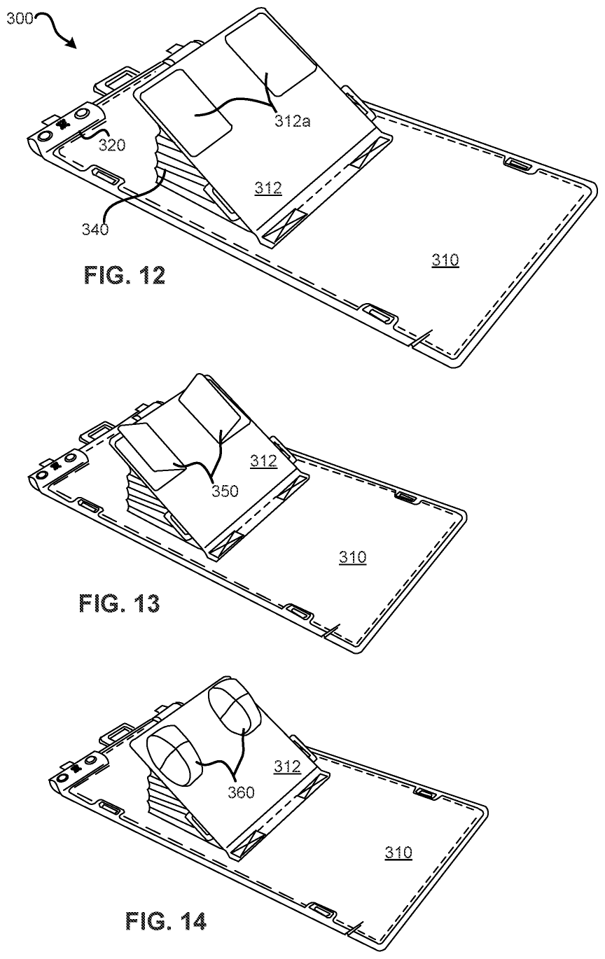

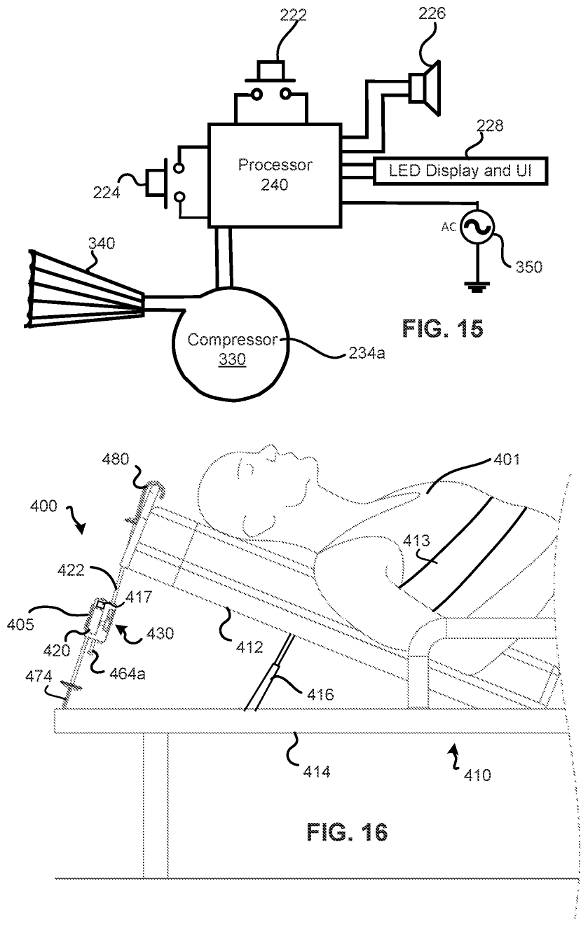

Referring now to FIGS. 10-12 and 15, there is shown a further embodiment of a device useful for performing elevated CPR. More particularly, a portable, head elevating CPR assist device 300 is provided that can be used in any location upon which the patient can be placed, including, but not limited to, the floor, a bed, a table, etc. The portable device 300 is ultra-lightweight and durable, enabling it to be carried to a patient's location, for use in performing automated, elevated CPR. In one particularly preferred embodiment, the device 300 is made from super lightweight, woven fabric encapsulating lightweight panels.

For example, in one particular embodiment, a base pad 310 of the device 300 is made using a super lightweight, thin nylon fabric covering honeycomb boards. This is not meant to be limiting, as other mechanisms for producing the lightweight, portable device 300 can be used. For example, the base pad 310 of the device 300 can be made using lightweight panels, such as carbon fiber and expanded aluminum core, aluminum composites with plastic cores, blow molded or rotationally molded plastic hollow panels and/or expanded structural foam, such as expanded polypropylene foam (EPP). In the present particular embodiment, the lightweight panel would be sandwiched between sheets of a structural woven and reinforced fabric, such as nylon, or ballistic nylon, as desired. Such a construction makes the device 300 lightweight and portable, yet sturdy for use.

As with the previously described embodiments, the device 300 additionally includes a control panel 320 substantially similar to the control panel 220 described in connection with the embodiments of FIGS. 6-9, with like numbered elements performing like functions. However, control panel 320 of the present embodiment additionally includes a compressor 330 configured to inflate a bellows 340 that elevates (i.e., increases an angle of elevation of) a backrest 312, which is hingedly fixed or otherwise pivotally attached to the base pad 310. The processor 240 of the control panel 320 is programmed to inflate the bellows 340 in accordance with a program for performing elevated CPR stored in a non-transitory memory associated with the processor 240. In the present particular embodiment, the device 300 is powered by AC power source 350, such as by a cord and power transformer connected to a wall outlet at the location of use. However, this is not meant to be limiting, as the control panel 320 could be powered by an internal battery, if desired. In the embodiment illustrated, a cord compartment 314 is included as part of the base pad 310, for storage of a power cord of the device 300.

Additionally, in the present preferred embodiment, the base pad 310 includes lifting handles 316, for lifting the device 300 when occupied by a patient. The backrest 312 is provided with LUCAS® handles 318, for attachment to a LUCAS® Chest Compression System. Please note that the invention is not meant to be limited to the use of LUCAS® handles, as other types of handles may be used or, if desired, handles 318 may be omitted. Velcro tabs 311 are provided, to secure the base pad in the closed position, when folded as shown in FIG. 11. Additionally, a carrying handle 315 is provided for use in transporting the device 300 in the folded configuration.

The backrest 312 can include pads 312a used for retaining a patient's head in a desired position, during use. Other types of head pads can be used on the backrest 312, as desired. For example, in the embodiment of FIG. 13, ratcheting head pads 350 are provided on the back rest 312. In the embodiment of FIG. 14, inflatable pads or pillows 360 are provided for holding the patient's head in place, during use. Additionally, the inflatable pillows 360 are configured to be inflated by the compressor 330, under control by the processor 240.

Referring now to FIGS. 16-18, there is shown a further embodiment of a head elevating CPR assist device 400 that can be secured as a retrofit part to a stretcher 410 and used for performing elevated CPR. In one preferred embodiment of the invention, the device 400 is motorized, but manually controlled using the buttons 405 disposed on the housing 402. Alternately, the device 400 can include a processor and stored elevated CPR program, for automatic control of the motor 420, for example, to elevate the backrest 412, in steps, at discrete angles (e.g., 10, 20, 30 degrees) automatically. A rack-and-pinion gear system 430 is engaged with the motor 420, for adjusting the angle of inclination of the backrest 412. More particularly, a strap or band 422 incorporates the rack 434 on a face thereof, whereas, operation of the pinion gear 436 serves to pull or release the strap 422, depending on the direction of the motor 420. A guide 432 may be provided to support the rack 434.

As the motor 420 turns, the length of the upper device strap 464 extending between the top of the housing 402 and the upper attachment element 480 changes, depending on the direction of operation of the motor 420. An excess amount 464a of the strap 464 can extend from the bottom of the housing 402, as illustrated in FIG. 16, depending on the length of the strap portion 464 extending outside the top of the housing 402. However, this is not meant to be limiting, as an excess amount 464a can be contained inside the housing 402, if desired, or can be arranged in another way, as desired.

In one particular embodiment, the lower device strap 454 has a fixed length, and the total overall length of the device 400 changes based on the lengthening or shortening of the strap 464, via operation of the motor 420 and gear system 430. Note that this is not meant to be limiting, as the lower device strap 454 could include the rack 434, instead of, or in addition to, the upper strap 464, as desired. The motor 420 can be powered by an internal battery (not shown) or by an external power source, as desired.

The head elevating CPR assist device 400 acts similarly to the device 100 of FIGS. 1-5, in that it attaches to a stretcher in which the locking mechanism for the backrest 412 has been released. For example, a strap (not shown) can be used to bias a backrest adjustment lever open, as discussed in connection with the embodiment of FIGS. 1-5, wherein the gas cylinder 416 biases the backrest 412 towards an elevated position. The device 400 is attached between a portion of the head end of the back rest 412, and the frame 414 of the stretcher, using attachment mechanisms 474 and 480. For example, the attachment mechanism 474 and/or 480 can be a hook or loopable strap, as desired, and the device 400 can be hooked and/or strapped to the respective portions of the stretcher, as desired. The device 400 operates to increase and decrease the angle of elevation of the backrest 412, thus changing the height of the head and thorax of a patient, such that the head of the patient is elevated and rests higher than the patient's torso. In the present illustrated embodiment, the motor 420 and gearing or gear train 430 operate to either shorten the length of the device 400 to pull the backrest 412 down (i.e., counter to the force applied by the gas cylinder 416) or to lengthen the device 400 to allow the gas cylinder 416 to raise the backrest 412 to the full extent permitted by the device 400. Note that other types of gearing or motorized movement can be used without departing from the scope or spirit of the present invention. If desired, the motor may be omitted, and other mechanical mechanisms used to engage the gear system to lengthen or shorten the device 400, such as a winding key or crank, and/or a pull tab at the end of the straps 464 and/or 454.

Additionally, the head elevating CPR assist device 400 can include indicator marks 484, 486, 488 on at least one extendible device strap (464 in the embodiment illustrated in FIGS. 16-18) that can be viewed through the mark viewing slots 416. In particular, in the embodiment of FIGS. 16-18, the motor 420 is operated using the desired directional button 405 until the desired mark 484, 486, 488 registers with the mark viewing arrows 414 in the viewing slots 416.

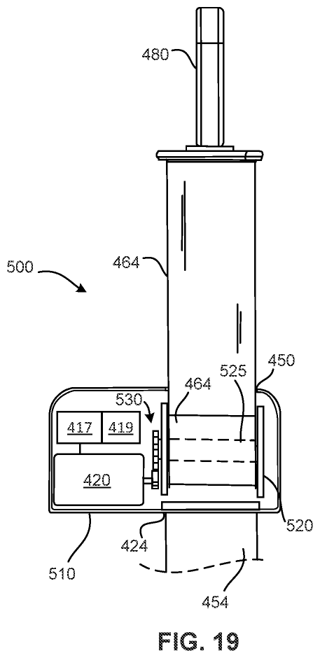

The head elevating CPR assist device 400 may be deployed between the stretcher frame 414 and the handle of the backrest 412 of the stretcher 410 to facilitate implementation of first, second and third stages of an elevated CPR procedure on the patient 401 as the head and torso of the patient recline at respective elevated positions at the first, second and third angles. Implementation of CPR on the patient 401 at these elevated positions may help relieve pressure on the brain of the patient 401. The device 400 may be helpful in retaining the upper or head portion of the backrest 412 at the elevated first angle (in the present example, corresponding to 10° of inclination), second angle (in the present example, corresponding to 20° of inclination) and third angle (in the present example, corresponding to 30° of inclination), respectively, with respect to the stretcher frame 414 as a patient 401 reclines on the backrest 412. A mechanism, such as strap 413, can be provided on the stretcher 410 for holding the patient and preventing the patient from sliding down when the maximum elevation is reached. A method for performing elevated CPR, as discussed above in connection with the embodiment of FIGS. 1-15, can similarly be performed using the device 400 by actuating the directional buttons 405, to control the length of the device 400 and select a particular angle of inclination of the head portion of the backrest 412. Note that other angles may be used, as desired, instead of, or in addition to, the above-described angles of inclination of 10°, 20° and 30°. Additionally, control of the motor 420 to achieve the desired angle of inclination can be performed by a processor 417 of the device 400, as described in connection with the embodiment of FIGS. 1-5. More particularly, the motor 420 can be controlled by an onboard processor 417 executing a pre-stored program (stored in onboard memory 419 of FIG. 19) for performing elevated CPR.

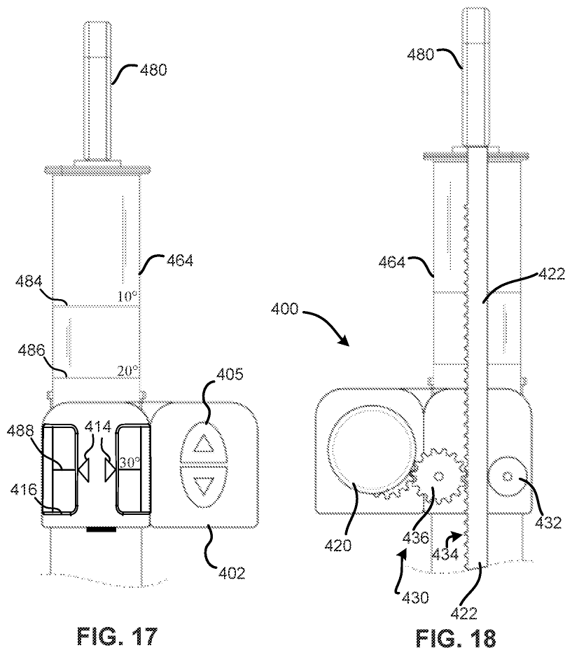

A further embodiment of a head elevating CPR assist device 500 is illustrated in FIG. 19. The device 300 can be used in the same manner as described in connection with the embodiment of FIGS. 16-18, above. However, in the present particular embodiment, the motor 420 does not use the rack and pinion gearing system 430 of FIGS. 16-18 to lengthen or shorten the device 500. Instead, an end portion of at least one of the of the straps 464 and/or 454 passes through a respective one of the upper strap slot 450 and/or lower strap slot 424 in the housing 510 and is mounted on a reel 520 and/or a spindle 525 in the housing 510. In the embodiment illustrated, the upper strap 464 passes through the slot 450 and is mounted on reel 520 and/or spindle 525, which may be spring-biased, as desired. Motor 420 can be used to rotate the reel 520 and/or spindle 525, to wrap the strap 464 or 454 thereon, or unwrap the strap 464 or 454 therefrom, to shorten or lengthen the device 500. A gear system 530 may be used to interface a spindle of the motor 420 to the spool 520. Alternately, the motor spindle may be used as the spindle 525, if desired. Directional buttons (405 of FIG. 17) can be used to control the direction of the motor 420, and thus, control whether the device 500 is lengthened or shortened. Otherwise, the device 500 operates as described in connection with the device 400 of FIGS. 16-118, in order to adjust the length of the device 500 to elevate the head and thorax of the patient to the desired angles for performing elevated CPR according to the methods described herein and/or in U.S. Pat. No. 10,350,137 to Lurie, incorporated herein, by reference. The motor 420 can be controlled by an onboard processor 417 executing a program pre-stored in non-transitory memory device 419 for performing elevated CPR.

Elevated or Heads Up CPR can be performed using any of the devices described herein, programmed according to the elevation timings disclosed in U.S. Pat. No. 10,350,137 to Lurie, previously incorporated herein, by reference, in its entirety. Other timing and elevation methods for performing elevated CPR may be used with the devices described herein, including those described in U.S. Pat. No. 9,750,661 to Lurie et al., filed on Jan. 14, 2016; U.S. Pat. No. 10,406,069 to Lurie et al., filed on May 22, 2018; United States Patent Application Publication No. 2016/0228326 to Lurie et al., filed on Apr. 20, 2016; United States Patent Application Publication No. 2018/0110667 to Freeman et al., filed on Oct. 19, 2017; and/or United States Patent Application Publication No. 2019/0175444 to Lurie et al., filed on May 20, 2016; those references being incorporated herein, by reference, in their entireties.

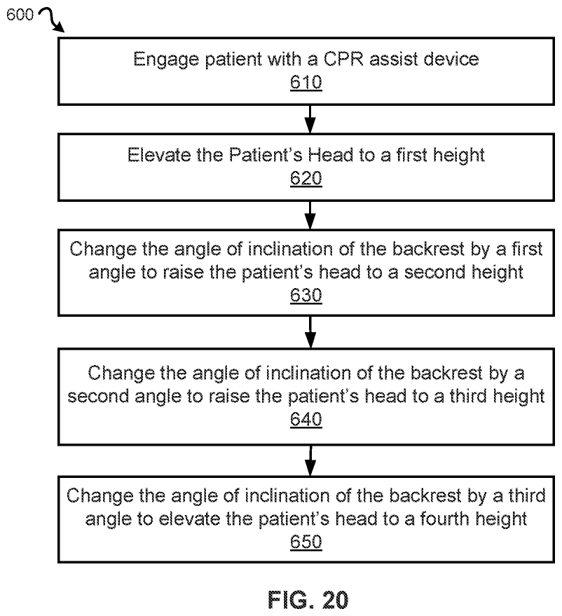

Additionally, referring now to FIG. 20, one particular method 600 for performing elevated CPR using an elevated CPR assist device, as described herein, is illustrated in FIG. 20. In the method 600 of the present invention, a patient typically starts in a horizontal position (i.e., a plane parallel to the floor), on a floor, stretcher, bed or other horizontal surface. A goal of the method of the present invention is to perform elevated or “head up” CPR on the patient, in which the head and thorax of the patient is elevated relative to the patient's torso. More particularly, a patient is first engaged with a CPR assist device, in accordance with the present invention. Step 610. More particularly, the patient is placed on a stretcher, bed or ambulance cot that includes a CPR assist device built in, or retrofitted by adding thereto, as described above in connection with FIGS. 1-9 and 16-19, or is supported on a portable, head elevating CPR assist device placed on a flat surface, as described in connection with FIGS. 10-15.

The patient's head is then raised to a slightly elevated position relative to the horizontal, and relative to the patient's torso. Step 620. More particularly, in this step, the patient's head is raised a minimum of 1.0 inch to a maximum of 1.5 inches above the horizontal for the first 30 seconds (1-30 seconds) of a 2 minute compressions cycle. The raising of the head relative to the horizontal (and the torso) in this step can be accomplished manually using a simple wedge or rolled up sheet between the horizontal plane (i.e., the patient bearing surface) and the patient's head. Other mechanisms for elevating the patient's head from 1.0-1.5 inches above the torso can also be used, without departing from the scope and spirit of the present invention. In another embodiment, the CPR assist device can be operated to provide the initial elevation in step 620.

After step 620, in the present exemplary method 600, the CPR assist device will operate to incline the backrest portion of the device 10 degrees over the next 30 seconds (i.e., 31-60 seconds) of the 2 minute compression cycle, thus further elevating the patient's head over the horizontal starting plane. Step 630. Note that, this operation of the CPR assist device to change the angle of inclination of the backrest will also simultaneously elevate a portion of the patient's torso.

After step 630, in the present exemplary method 600, the CPR assist device will operate to incline the backrest portion of the device to 20 degrees relative to the horizontal plane over the next 30 seconds (i.e., 61-90 seconds) of the 2 minute compression cycle in order to further raise the patient's head above the horizontal starting plane. Step 640. As noted above, the torso will be elevated, as well, but the head will be elevated slightly higher than the torso.

After step 640, in the present exemplary method 600, the CPR assist device will operate to incline the backrest portion of the device to 30 degrees relative to the horizontal plane over the next 30 seconds (i.e., 91-120 seconds) of the 2 minute compression cycle, thus elevating the patient's head to its maximum position. Step 650. In one particular embodiment, at an angle of inclination of the backrest to 30 degrees relative to the horizontal starting plane, the patient's head will be raised to a maximum of 6 inches above the horizontal starting plane. A restraint mechanism can be employed to prevent the patient from sliding down the backrest at the highest point of elevation. In one particular example, the restraint mechanism includes a strap (113 of FIGS. 3A-3B; 413 of FIG. 16) that is, preferably, positioned under the patient's armpits in use.

Accordingly, while a preferred embodiment of the present invention is shown and described herein, it will be understood that the invention may be embodied otherwise than as herein specifically illustrated or described, and that within the embodiments certain changes in the detail and construction, as well as the arrangement of the parts, may be made without departing from the principles of the present invention as defined by the appended claims.

We claim:

1. An elevated CPR assist device configured for elevating a head and thorax of a patient on a patient support that includes a backrest movable relative to a base, the elevated CPR assist device comprising:
a device housing;
a strap extendible from said device housing;
an attachment element at an end of said strap distal from said device housing, said attachment element configured for attachment to a portion of the patient support;
a mechanism disposed in said device housing and configured to change a length of said strap extending from said device housing;
a processor disposed in said housing, said processor controlling said mechanism to lengthen or shorten an amount of said strap extending from said housing according to a program for performing elevated CPR being executed by said processor; and
a user interface accessible from said device housing including at least one user activated element configured to initiate said program for performing elevated CPR,

2. The device of claim 1, wherein said device housing is configured for attachment to one of the backrest or the base and the attachment element is attached to the other one of the backrest or the base.

3. The device of claim 1, wherein the mechanism includes a motor.

4. The device of claim 3, wherein the mechanism further includes a reel driven by the motor, a housing end of the strap being attached to said reel, said motor operable to drive said reel to wind said strap onto said reel or unwind said strap from said reel,

5. The device of claim 4, further comprising a gear train disposed between said motor and said reel, said reel being driven indirectly by said motor via said gear train.

6. The device of claim 3, wherein the mechanism includes a rack-and-pinion gear system engages with the motor for adjusting the angle of inclination of the backrest.

7. The device of claim 1, wherein said user interface includes at least one indicator for providing audible or visual feedback, controlled by said processor

8. A method for performing elevated CPR on a patient arranged on a patient support of a stretcher including an elevated CPR assist device according to claim 1, the method comprising the steps of:
initiating the program for performing elevated CPR on the CPR assist device using the user interface; and
performing elevated CPR on the patient according to the program.

9. The method of claim 8, wherein the elevated CPR assist device is a self-contained unit designed to be added to the stretcher, and the method further includes the steps of:
securing the device housing to one of a backrest or a base of the stretcher;
securing the attachment element to the other one of the backrest or the base of the stretcher; and
releasing a locking mechanism of the backrest of the stretcher.

10. An elevated CPR assist device configured for elevating a head and thorax of a patient on a patient support that includes a backrest movable relative to a base, the elevated CPR assist device comprising:
a device housing configured for attachment to at least one of the backrest or the base;
at least one strap extendible from said device housing;
an attachment element at an end of said at least one strap distal from said device housing, said attachment element configured for attachment to the other one of the backrest or the base;
a mechanism disposed in said device housing and configured to change a length of said at least one strap extending outside said device housing between said device housing and said attachment element, said mechanism including a motor;
a user interface accessible from said device housing including at least one user activated element configured to operate said motor.

11. The elevated CPR assist device of claim 10, wherein said mechanism is controlled manually using buttons of said user interface for actuating said mechanism to adjust the angle of elevation of the backrest.

12. The elevated CPR assist device of claim 10, wherein said mechanism is controlled automatically by an onboard processor executing a pre-stored program for performing elevated CPR.

13. The elevated CPR assist device of claim 10, wherein said mechanism includes at least one gear mechanically connected with said motor, an angle of inclination of the backrest being changed relative to the base based on rotation of the at least one gear.

14. The elevated CPR assist device of claim 10, being a separate unit configured to be attached to a stretcher.

15. An elevated CPR assist system including:
an elevated CPR assist device according to claim 10; and
a stretcher;
said elevated CPR assist device being connected between the stretcher base and the backrest.

16. A method of performing elevated CPR, comprising the steps of:
providing an elevated CPR assist device according to claim 10;
attaching the elevated CPR assist device to a stretcher; and
operating the motor to change the angle of inclination of the backrest relative to the stretcher base.

17. A portable elevated CPR assist device, comprising:
a lightweight base pad;
a backrest pivotably attached to said lightweight base pad;
a mechanism attached between said backrest and said lightweight base pad, said mechanism operable to pivot said backrest relative to said lightweight base pad, in order to change the angle of inclination of the backrest relative to said lightweight base pad.

18. The portable elevated CPR assist device according to claim 17, wherein said mechanism includes an inflatable bellows.

19. The portable elevated CPR assist device according to claim 17, wherein said lightweight base pad includes a lightweight panel enclosed in a woven fabric.

20. The portable elevated CPR assist device according to claim 19 wherein the lightweight panel comprises at least one of a honeycomb board, carbon fiber with an expanded aluminum core, an aluminum composite with a plastic core, a blow molded plastic hollow panel, a rotationally molded plastic hollow panel, or expanded structural foam.
